(19) United States
(12) Patent Application Publication
(10) Pub. No.: US 2024/0398577 A1
(43) Pub. Date: Dec. 5, 2024

(54) Modular Humeral Component for Use in a Universal Shoulder Prosthesis System

(71) Applicant: Raphael S.F. Longobardi, LLC, Old Tappan, NJ (US)

(72) Inventor: Raphael S.F. Longobardi, Old Tappan, NJ (US)

(21) Appl. No.: 18/686,413

(22) PCT Filed: Sep. 1, 2022
(86) PCT No.: PCT/US2022/042345

§ 371 Date: Feb. 24, 2024

Related U.S. Application Data
(60) Provisional application No. 63/239,862, filed on Sep. 1, 2021.
(60) Provisional application No. 63/281,644, filed on Nov. 20, 2021.
(60) Provisional application No. 63/391,247, filed on Jul. 21, 2022.

(51) Int. Cl. A61F 2/40; A61F 2/30

(52) U.S. Cl. CPC A61F 2/4014; A61F 2002/30329; A61F 2002/30604; A61F 2002/30736

(57) ABSTRACT

A universal shoulder prosthesis system includes a baseplate, a shoulder base, which may be a baseplate, and modular components that are exchangeable between ta-TSR and r-TSR. The system may further include wedges for use with the baseplate when the baseplate is implanted on the glenoid side. An example of a modular component is a modular humeral component (500) for use in ta-TSR that includes an outer shell (510) and an inner head (520) that fits into the outer shell (510) and attaches to the shoulder base. In at least one embodiment, there is a means for engagement between the outer shell (510) and the inner head (520), which may be a ring protrusion (518A) encircling an interior cavity (514A) of the outer shell (510A) that fits into a channel (529) around the exterior of the inner head (520A).

This PCT application claims priority to U.S. Provisional Patent App. Nos. 63/239,862, filed on Sep. 1, 2021; 63/281,644, filed on Nov. 20, 2021; and 63/391,247, filed on Jul. 21, 2022, which are all incorporated by reference.

### I. FIELD OF THE INVENTION

The invention relates to a modular humeral component and/or a full or partial wedge for use in a modular shoulder prosthesis system that provides for flexibility in shoulder replacements and allows for a more efficient switch for a patient between a traditional anatomic Total Shoulder Replacement (ta-TSR) to a reverse Total Shoulder Replacement (r-TSR). The modular humeral component includes an inner head onto which is attached a humeral shell that, in at least one embodiment, is able to rotate about the inner head and, in at least one embodiment, is able to slide along the surface of the shell along an arc. The full or partial wedge allows use of a standard baseplate in situations where there is a bony deformity in the patient's glenoid, and, in at least one embodiment, the wedge and the baseplate are made of metal for use on the glenoid side.

### II. BACKGROUND OF THE INVENTION

TSRs have evolved over the last 70 years, with the greatest degree of its evolution occurring within the past 20 years. The understanding of the complexity of the shoulder has resulted in the ability to better treat the multiple conditions that afflict the shoulder. Glenohumeral arthritis ranges from simple to complex due to etiology and deformity. Post traumatic glenohumeral arthritis, along with the deformity of both the glenoid and humeral head present challenges for the shoulder arthroplasty surgeon. Similarly, the problem of rotator cuff deficiency and rotator cuff arthropathy has resulted in the development of treatment and prosthetic designs specific to address the loss of the main motors of the shoulder.

Currently, there are two types of TSR—traditional anatomic total shoulder replacement (ta-TSR) and reverse total shoulder replacement (r-TSR). Ta-TSR utilizes resurfacing of the humeral head and glenoid in the setting of an intact and functioning rotator cuff. Glenohumeral arthritis has been treated with ta-TSR, the current gold standard being the resurfacing of the humeral head with a stemmed or metaphyseal component along with a replacement of the humeral head articular portion with a cobalt-chromium (Co-Cr) implant. Modularity of the humeral components allows for appropriate sizing of the head in diameter and thickness to match the resected articular surface of the patient.

### III. SUMMARY OF THE INVENTION

Currently, all commercial ta-TSR, and r-TSR require removal of all components, especially the glenoid components, when revising. In the specific case of revision from ta-TSR to r-TSR, a universal system allows for continued use of the glenoid and humeral baseplates, with removal and substitution of the ta-TSR articulating surfaces [glenoid and humeral head] with those of the r-TSR articulating surfaces [glenosphere and humeral cup]. A problem arises when a patient has anterior instability leading to potential humeral head sublocation anteriorly. This condition typically requires greater retroversion of the components, which requires changes in the version of the humeral head osteotomy, or further bone removal from the glenoid to provide a greater angle for the glenoid component to prevent anterior instability. Instead, the purpose of the components in at least one embodiment are to correct bony deformity, version issues and instability by building into the all-metal glenoid components corrections. In this case of anterior instability, the use of a 7-degree anterior build up would be utilized instead of a standard glenoid component to prevent further anterior instability.

A modular shoulder prosthesis system allows the surgeon to achieve either exchanges, humeral or glenoid component, without an extravagant amount of equipment to be used, or more complex operative procedures to be performed. A truly versatile and modular system allows for a baseplate to accept either a traditional anatomic humeral component or a reverse humeral cup, without compromising long term security and function.

In at least one embodiment, there is a modular humeral component for use in a modular shoulder prosthesis system having a shoulder base, e.g., a baseplate or a humeral stem with (or without) notches and/or attachment points, the modular humeral component including: an inner head having a base, a dome on the base, an optional plug extending away from the base, and an optional flange extending away from a peripheral edge of the base where the flange is configured to fit over the shoulder base; and a shell configured to fit over the inner head, the outer shell having a body having an exterior surface configured for engagement with a concave surface of a glenoid component, the body defining an interior cavity configured to receive the inner head, and optionally an interiorly facing ledge along a bottom of the body and/or other complementary engagement feature to engage the inner head.

In a further embodiment, the ledge hooks over the flange. In a further embodiment to the embodiment of the previous paragraph, a top surface of the ledge is beveled in a direction of its free edge. In a further embodiment, a bottom exterior edge of the flange and/or inner head is beveled to correspond to the bevel of the ledge. In an alternative or further embodiment, the inner cavity of the shell includes a receiving cavity having an outer inward directed bevel edge (in an alternative to the ledge), an engagement protrusion to snap into a receiving channel of the inner head, and a concave region for receiving the dome of the inner head. The ledge and the engagement protrusion are examples of an engagement means for attachment of the shell to the inner head particularly where the engagement means in in the interior cavity.

In a further embodiment to the previous embodiments, when the modular humeral component is installed on the shoulder base, a gap between a free edge of the ledge of the inner circumference of the shell and the shoulder base is approximately 1 mm. In a further embodiment to the previous embodiments, the optional plug is tapered in a direction from its top to its bottom. In a further embodiment to the previous embodiments, the dome is a hemi-spherical dome and the interior cavity of the shell is hemi-spherical, the shell is configured to slide and/or rotate relative about the dome to allow for dual mobility between the shell and the inner head such that any pivoting between them is limited by the ledge and/or the bottom of the shell. In a further embodiment to the previous embodiments, the optional flange includes a pair of interior protrusions extending down from the base, the protrusions configured to engage notches in a mounting surface of the shoulder base. In a further embodiment to the previous embodiments, the inner head includes cobalt-chromium and the shell includes a high-density polyethylene. In a further embodiment, the inner head may be manufactured and bonded to the outer shell to provide a single fabricated component.

In at least one embodiment, there is a wedge for use on the glenoid side in a modular shoulder prosthesis system having a baseplate with notches and/or attachment points, the wedge including: a body having a mounting surface and a bone facing surface angled relative to the mounting surface, a plurality of fastener holes passing therethrough from the bone facing surface to the mounting surface, and a central opening passing therethrough from the bone facing surface to the mounting surface configured to receive a stem of the baseplate, and optionally the central opening has an interference fit with the stem. In a further embodiment, the angle between the mounting surface and the bone facing surface is 10 degrees, 25 degrees, between 10 and 30 degrees, between 15 and 30 degrees, and between 10 and 25 degrees. In a further wedge embodiment, each of the plurality of fastener holes includes a shoulder proximate to the mounting surface. In a further wedge embodiment, the number of fastener holes is 2, 3, or 4. In a further wedge embodiment, the wedge is a partial wedge configured to cover a portion of the base of the baseplate, and optionally the central opening is not fully encircled by the body. In a further wedge embodiment, the wedge is a full wedge. In a further wedge embodiment, a height on one side is approximately 0.25 mm. In a further wedge embodiment, there are a number of fasteners equal to the number of fastener holes, and the fasteners include screws, bolts, and/or pegs. In a further wedge embodiment, the wedge has a plurality of anchoring holes passing from the bone facing surface to the mounting surface to be aligned with the perimeter mounting holes of the baseplate to allow the anchoring mechanisms to pass therethrough to provide additional anchoring points for the prosthesis to the patient's bone.

In at least one embodiment, the 10-degree posterior angulation of the wedges facilitate reconstruction of idealized glenoid version in the anterior-posterior and sup-inf planes and/or correction of the bony deformity of the glenoid. The wedges mitigate the need for removal for a revision to occur. In at least one embodiment, the glenoid component in traditional TSR with 7-degree anterior build-up may address anterior instability concerns.

In at least one embodiment, there is a modular shoulder prosthesis system includes a baseplate and/or a humeral stem and any of the modular humeral components described above and/or any of the wedges described above.

### IV. BRIEF DESCRIPTION OF THE DRAWINGS

Any cross-hatching or shading present in the figures is not intended to identify or limit the type of material present for the element shown in cross-section. In figures that include multiple elements shown in cross-section, the cross-hatching will be different directions for the different elements. Some features are illustrated as dashed or phantom lines in the figures.

FIGS. 1A-1D illustrate a top perspective view, a bottom perspective view, a side view, and a cross section-view view taken through a diameter of an example baseplate.

FIGS. 2A-2D illustrate a top view, a top perspective view, a bottom perspective view, and a bottom view of another example baseplate.

FIGS. 3A-3C illustrate a full wedge for use with a baseplate like the universal baseplates illustrated in FIGS. 1A-2D. FIGS. 3A-3C illustrate a top view, a top perspective view, and a substantially bottom view, respectively. FIG. 3D illustrates a top perspective view of another full wedge embodiment. FIG. 3E illustrates a side view of a full wedge attached to a baseplate. FIG. 3E illustrates a side view of a full wedge attached to a baseplate.

FIGS. 4A-4C illustrate a partial wedge for use with a baseplate like the universal baseplates illustrated in FIGS. 1A-2D. FIGS. 4A-4C illustrate a top view, a top perspective view, and a bottom perspective view, respectively. FIGS. 4D-4F illustrate the partial wedge illustrated in FIGS. 4A-4C with the baseplate illustrated in FIGS. 2A-2D. FIGS. 4A-4C illustrate a top exploded perspective view, a side view, and a bottom view, respectively. FIG. 4G illustrates a top exploded perspective view of another partial wedge embodiment.

FIGS. 5A-5F illustrate a modular humeral component. FIGS. 5A-5B illustrate the modular component with the baseplate illustrated in FIGS. 2A-2D in a sectional view illustrating interior features and an exploded partial cross-sectional view taken along a diameter between the lower notches of the baseplate, respectively. FIGS. 5C-5D illustrate a cross-section view of a shell along a diameter taken from side to side and a bottom view of the shell, respectively. FIGS. 5E-5F illustrate a side view and a bottom view of the inner head, respectively.

FIGS. 6A-6K illustrate another modular humeral component. FIG. 6A illustrates an exploded, cross-sectional view of a modular humeral component and an example baseplate. FIGS. 6B and 6C illustrate a top perspective view and side view of a shell. FIGS. 6D-6G illustrate a top view, a perspective view, a side view, and a bottom view of an inner head. FIGS. 6H-6K illustrate an assembled humeral head with a bottom perspective view, a side view, a side phantom view, and a bottom view.

### V. DETAILED DESCRIPTION OF THE DRAWINGS

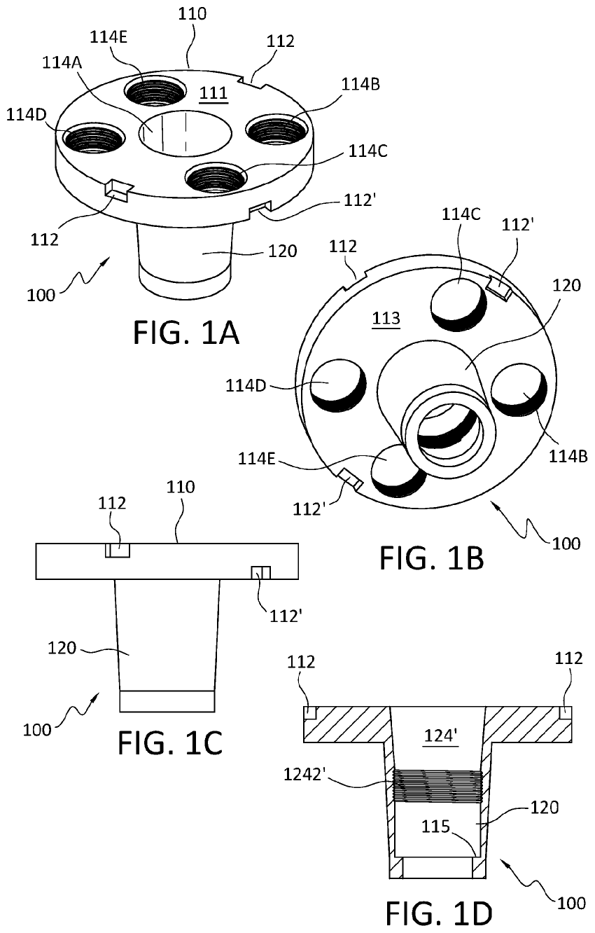

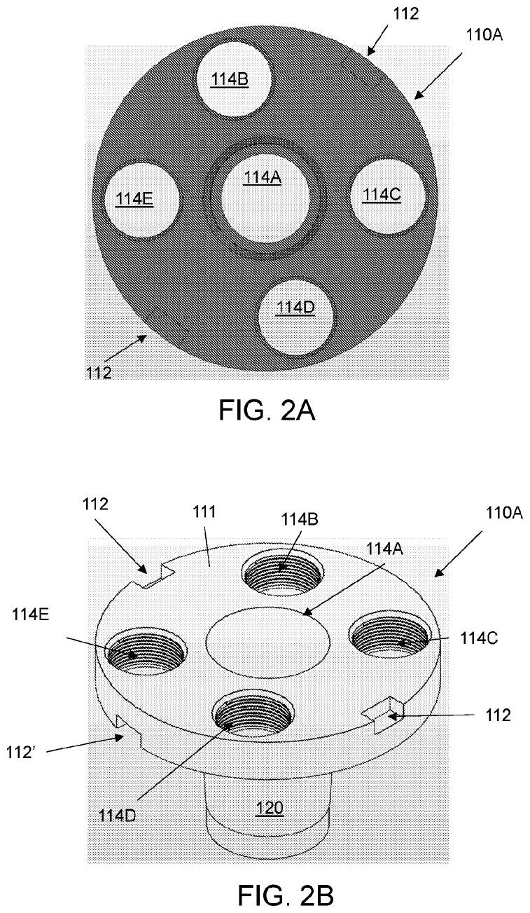

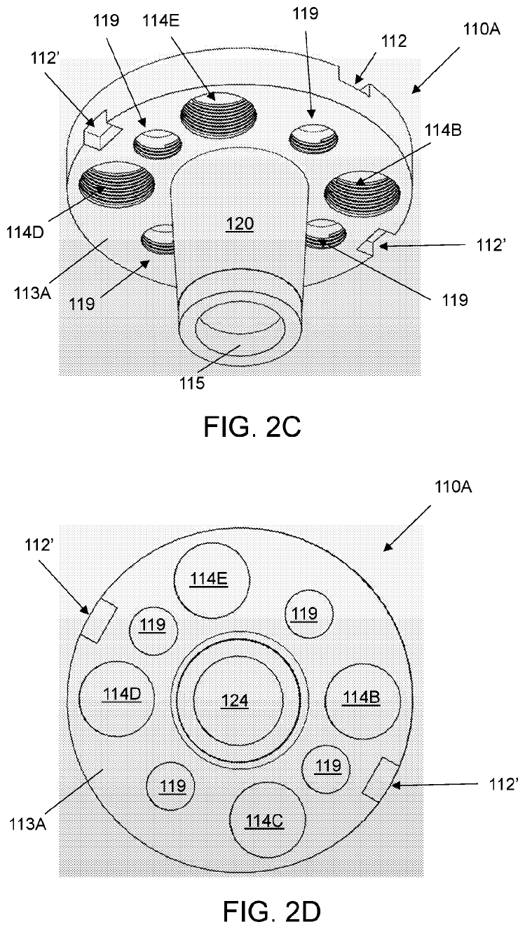

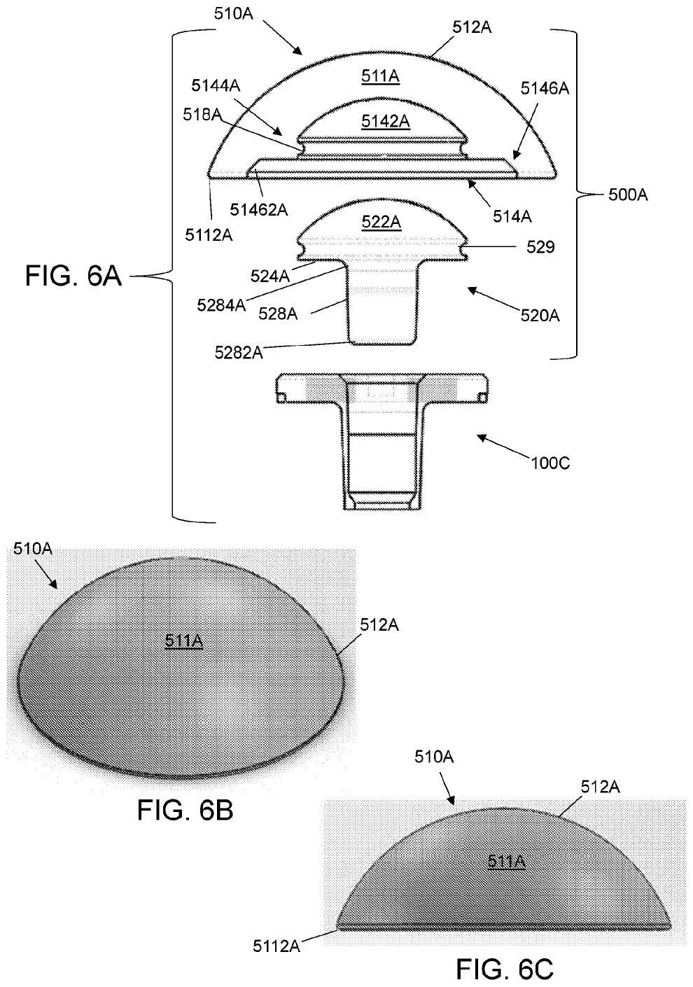

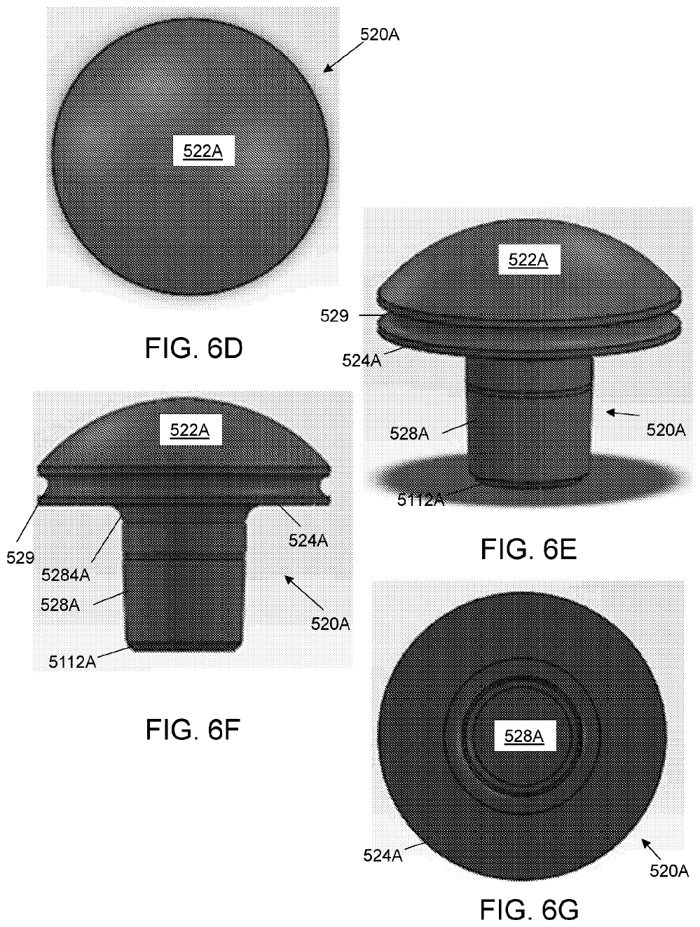

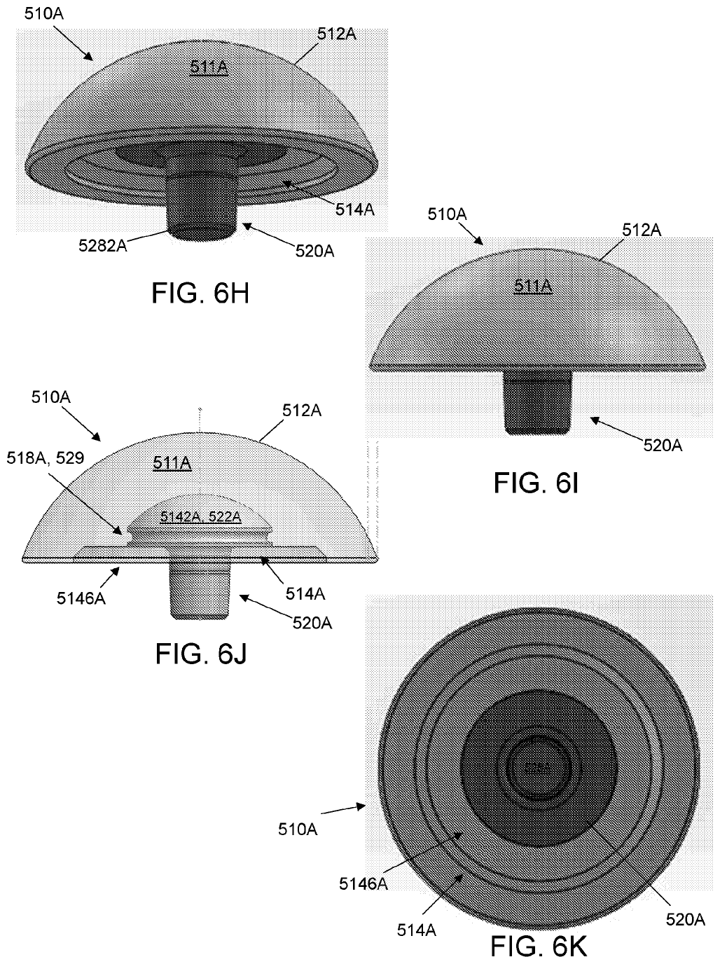

The invention in at least one embodiment includes a humeral modular component for use with baseplates disclosed in U.S. Pat. No. 10,583,012, issued on Mar. 10, 2020, and PCT Application No. PCT/US21/20492, published as WO 2021/178418 A1 on Sep. 10, 2021 and filed on Mar. 2, 2021, which are hereby both incorporated by reference for their teachings regarding different baseplates. FIGS. 1A-2D illustrate two different examples of baseplates. FIG. 6A illustrates another baseplate that includes a receiving cavity with a slight Morse taper present in it for receiving the humeral head base. Such a cavity may be present in the above incorporated baseplates.

The baseplate has a mounting surface for engagement of a modular component. The mounting surface refers to the substantially planar surface of the baseplate opposite the glenoid (or humerus) after being implanted. The modular humeral component illustrated and described in this disclosure is an example of a modular component. The attachment in at least one embodiment between the modular component and the baseplate is through, for example, a Morse taper, which may be a dual threaded Morse taper that is axially located with reference to the baseplate. In an alternative embodiment with or without the Morse taper, a torque limiting fastener, such as a screw or a bolt, is used to further secure the modular glenoid component to the baseplate by engaging the baseplate and/or the central attachment mechanism anchored in the patient's glenoid/humerus. In a further embodiment, the attachment is facilitated with a threaded connection where the modular component is screwed into the baseplate.

FIGS. 1A-ID illustrate a baseplate 100. The illustrated baseplate 100 includes a base 110 and a stem 120 extending from the base 110. Although the base 110 is illustrated as being circular, the base 110 may be elliptical, oval, or other suitable shapes; in such an embodiment, the modular component may be shaped to match. Examples of the diameter of the base 110 include between 20 mm and 35 mm, 20 mm, 25 mm, 30 mm, and 35 mm. Examples of the thickness of the base 110 include between 5 mm and 15 mm (with or without the end points), 5 mm, 7 mm, 10 mm, 12.5 mm, and 15 mm. In at least one embodiment, the baseplate 100 is made from an ingrowth trabecular metal over a metal core of, for example, steel, titanium or a combination of the two. The ingrowth trabecular metal facilitates bone ingrowth into the baseplate 100, for example to increase the strength of the connection between the bone and the baseplate 100 and the respective interface shear strength overtime.

Although the stem 120 illustrated as a cylinder with a slight taper, in at least one embodiment, the stem 120 has tapered sides to match the modular component plugs. In a further embodiment, the stem 120 is substantially cylindrical. FIG. 6A illustrates a slightly different stem 100B.

The base 110 includes amounting surface 111 on a side opposite of an implantation surface 113, which faces the bone after implantation and from which the stem 120 extends. In at least one embodiment, the mounting surface 111 is substantially planar. The plane defined by the mounting surface 111 is approximately parallel to the resection plane after implantation.

The illustrated base 110 of FIGS. 1A-1C includes a pair of opposed leverage notches 112, 112 extending down from outer circumferential sides of the mounting surface 111, for example on the anterior and posterior central exterior edge (or side) of the base 110, which in at least one embodiment provides better access to the notches for removal of the modular component although the notches may be rotated 90 degrees from the anterior and posterior sides of the base 110. The notches 112, 112 are configured to be accessible from the mounting surface 111. In at least one embodiment, the notches 112, 112 provide (or are configured to have) a leverage point to facilitate separation of the mounted modular component from the baseplate 100 when the baseplate 100 is implanted. The notches 112, 112 have sufficient width and depth to receive an instrument in which to pry the mounted modular component from the baseplate 100. Although two notches 112, 112 are illustrated, additional notches could be added to the base 110. In a further embodiment, each of the notches 112, 112 receive a protrusion depending from a base of the modular component, for example to increase the strength of the connection between the base 110 and the modular component. In at least one embodiment when additional notches are present, the modular component may be rotated relative to the base to engage the two notches that provide a preferred orientation for engagement of the modular humeral component by the modular component. In a further embodiment, a space will be defined by the notch 112 and the protrusion to receive an instrument to pry the mounted modular component from the baseplate 100. In at least one embodiment, the leverage notches 112, 112 are omitted, while in another embodiment the leverage notches are the attachment points for fingers of the instrument.

The illustrated base 110 includes a pair of attachment points, which are illustrated as notches 112′, 112′. In at least one other embodiment illustrated in FIGS. 1A-1C and 2B-2C, the attachment points 112′, 112′ are position at 7 o'clock and 1 o'clock when the notches 112, 112 are at the anterior and posterior walls (i.e., 3 o'clock and 9 o'clock). In an alternative embodiment, the attachment points are aligned with the notches. The notches extend up from outer circumferential sides of the bottom surface 113 from which the stem 120 extends. The notches 112′ are present on the outer circumferential sides of the base 110. Both types of attachment points are accessible from the exterior of the baseplate 100. In at least one embodiment, the attachment points 112′, 112′ have sufficient width and depth to engage with an implanting/extracting instrument. In at least one further embodiment, the attachment points 112′, 112′ are configured to match the shape of the instrument used for implanting and/or extracting the baseplate 100 including the cross-section and/or depth of a finger of the instrument. The instrument fingers are inserted into the attachment points 112/112 to assist with the implantation of the baseplate 100 onto the glenoid/humerus and, if necessary, is used to extract the baseplate 100 from the glenoid/humerus.

The illustrated base 110 includes five mounting passageways 114A-114E passing from the mounting surface 111 to the implantation surface 113 (or through the stem 120) with an axially centered passageway 114A and four evenly spaced perimeter passageways 114B-114E around the mounting surface 111. The passageways 114A-114E may include a shoulder at or near the bottom of the passageway on which a screwhead, which screw is an example of an attachment mechanism, will make contact after insertion into the baseplate 100. Although five passageways are illustrated, the number of passageways could be reduced, including omission of the perimeter passageways, or increased. In at least one embodiment, the central passageway 114A defines a chamber 124 for receiving a modular component plug. In at least one embodiment, the passageways 114B-114E are offset from the notches 112, 112 as illustrated, for example, in FIGS. 1A, 1B, and 2A-2D. For example, passageway 114B might be at approximately 1 o'clock or 2 o'clock while passageway 114E might be at approximately 10 o'clock or 11 o'clock if the notches 112, 112 are at 3 o'clock and 9 o'clock, respectively.

In at least one embodiment, one or more variable angle locking screws are used to attach the baseplate 100 to the patient's glenoid bone. Examples of screw diameters include 4.5 mm to 5.0 mm and in a further example including the end points of that range, and more particularly 5.0 mm. Although there are five passageways illustrated, during a particular procedure, all five passageways may not be utilized. In at least one embodiment, the flexibility in which passageways 114A-114E to use and the variable angle locking screws provides flexibility to the orthopedic surgeon in securing the baseplate 100 to the patient's bone. Examples of locking screw angles includes between 20 degrees and 30 degrees (with or without the end points) or perpendicular to the base 110.

In at least one embodiment, the central axial passageway 114A passes from the base 110 into and through the stem 120 to allow for the top of the locking screw to be deeper into the baseplate 100 and to provide the chamber 124′ for receiving a plug, e.g., the Morse taper, of the modular component being mounted onto the baseplate 100. FIG. 1D illustrates the chamber 124′ having receiving screw threads 1242′ for engaging a torque limiting fastener. As referenced above, the torque limiting fastener may engage a locking screw instead or in addition to other areas of the chamber 124′.

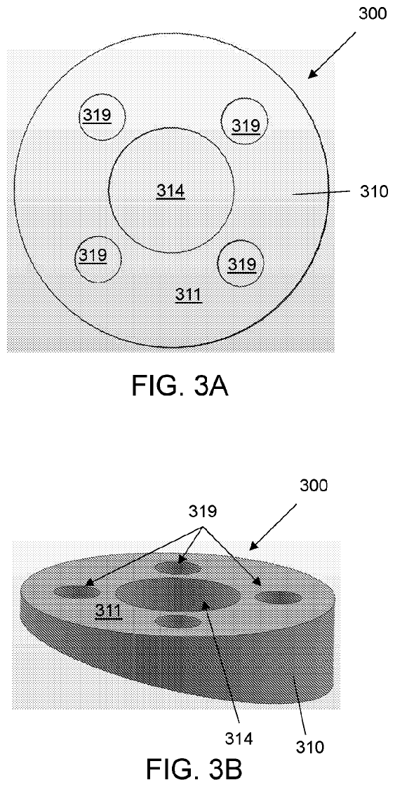

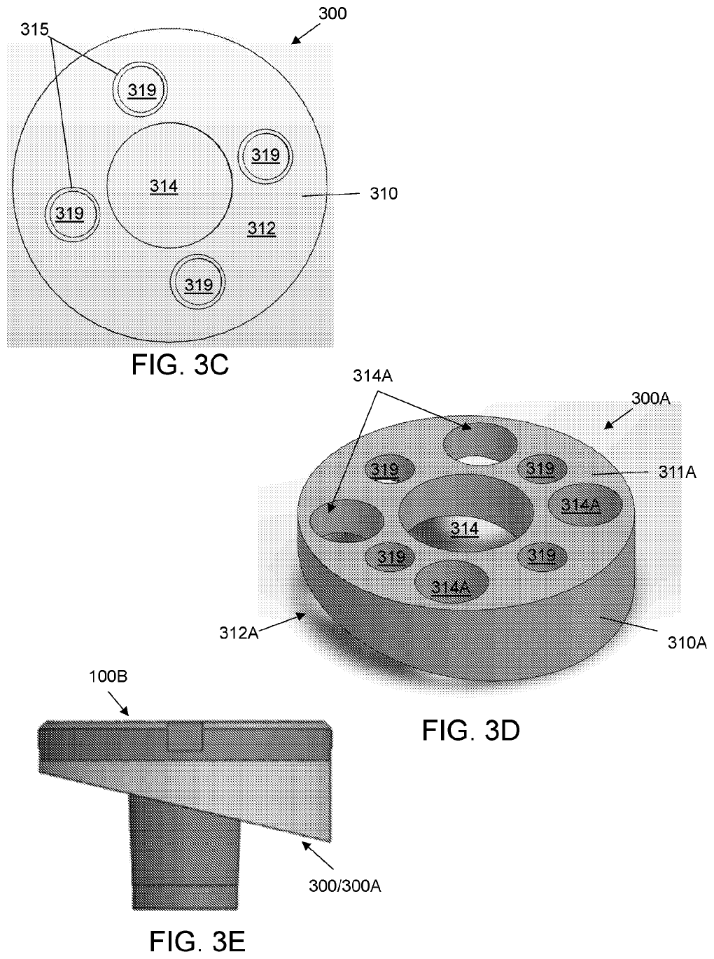

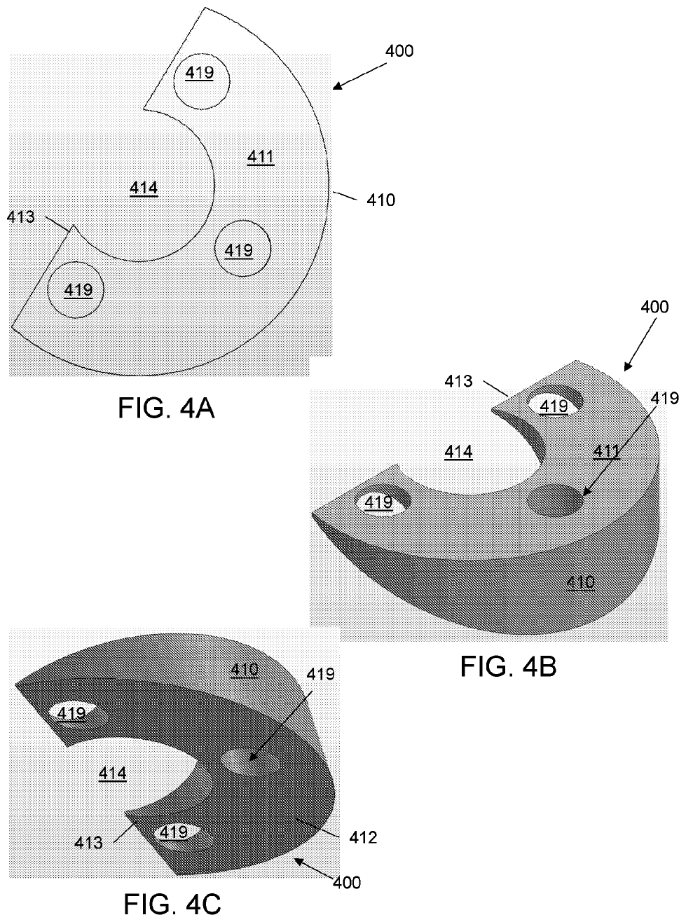

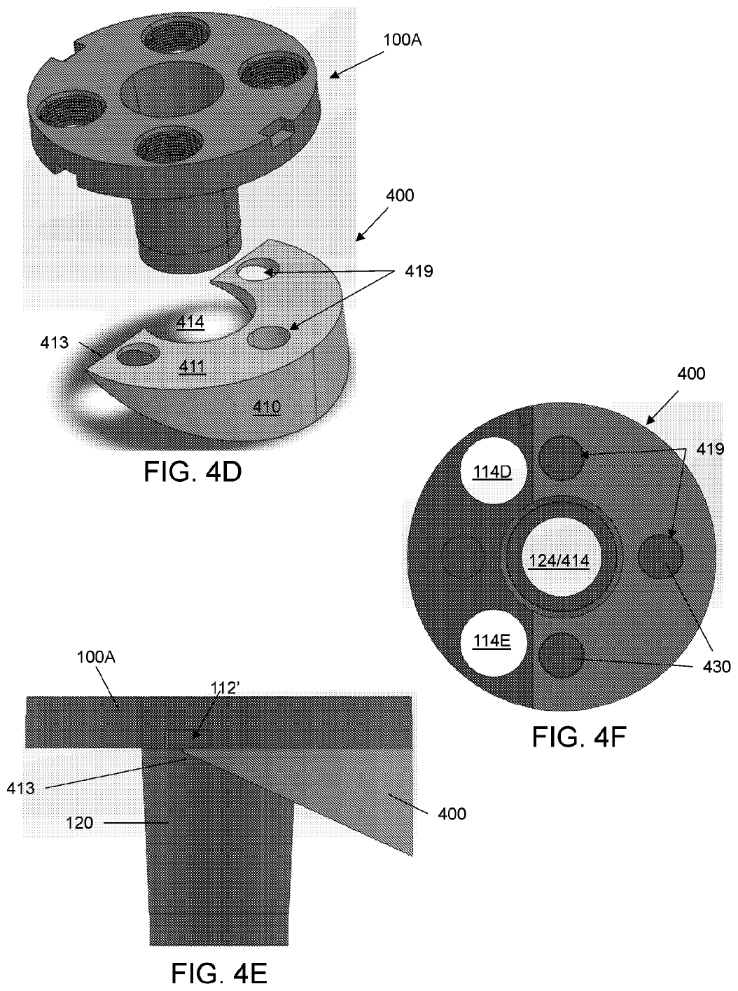

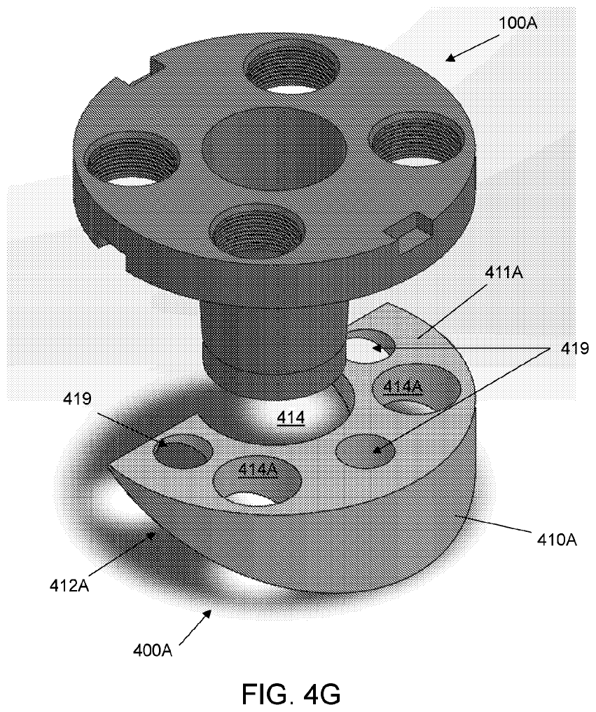

FIGS. 2A-2D illustrate a baseplate 100A with a base 110A having four recessed cavities 119 in its bottom surface 113A for engagement with a fastener to optionally attach a full wedge 300 illustrated, for example, in FIGS. 3A-3C or a partial wedge 400 illustrated, for example, in FIGS. 4A-4F. The illustrated baseplate 100A shares a variety of features with the baseplates illustrated in FIGS. 1A-ID. In an alternative embodiment, the number of recessed cavities is two or three (although not illustrated). The illustrated recessed cavities 119 may be included as part of any baseplate illustrated in the figures. As illustrated, the recessed cavities 119 are offset from the mounting passageways 114B-114E and the stem 120. In at least one embodiment, the recessed cavities include a threaded section for engagement of a fastener as illustrated in FIG. 2B, although in an alternative embodiment the threaded section may be omitted and reliance made on a fiction engagement.

FIGS. 3A-4G illustrate full wedges 300, 300A and partial wedges 400, 400A that may be used in conjunction with the described baseplates and/or the modified baseplate 100A illustrated in FIGS. 2A-2B. The wedges are configured for use on the glenoid side of a shoulder prosthesis. The full wedge 300, 300A and the partial wedge 400, 400A provide the ability to place a mounting surface 111 at an angle, for example from the glenoid resection plane. One of ordinary skill in the art should appreciate that the presence of a wedge is potentially advantageous for addressing bone deformities of the glenoid (or humerus) while providing a secure attachment, for example, of the baseplate 100 or 100A to the patient's bone. In embodiments that uses one of the wedges, the attachment points will remain the notches 112′ on the opposing sides of the baseplates. The wedges facilitate reconstruction of idealized glenoid version in the anterior-posterior and sup-inf planes. The wedges mitigate the need for removal for a revision to occur.

FIGS. 3A-3C illustrate an example of a full wedge 300 having a body 310 with a central hole 314 and a plurality of fastener holes 319 passing therethrough. In at least one embodiment as illustrated in FIG. 3C, the fastener holes may include a shoulder 315 against which the fastener 430 (illustrated in FIG. 4F) such as a bolt, a screw, or a peg can abut to secure the wedge 300 to the baseplate, e.g., baseplate 100A. The fastener holes 319 are configured to align with the recessed cavities 119. The presence of the fastener holes 319 allows for the wedge 300 to be secured to the baseplate 100 or 100A prior to insertion in the patient. In an alternative embodiment, the fastener holes 319 are aligned with the perimeter mounting holes 114B-114E of the baseplate 100-100C to allow for anchor mechanisms to pass therethrough for additional anchor points for the prosthesis to the patient's bone.

The central hole 314 passes through the wedge body 310 to allow for the stem 120 to pass through for anchoring the system to the patient's bone. In at least one embodiment, the central hole 314 has an interference fit with the stem 120.

The wedge body 310 includes a mounting surface 311 onto which the attached baseplate abuts and/or is against. The opposed surface to the mounting surface 311 is a bone facing surface 312 that is designed to abut and/or be against the patient's bone. The bone facing surface 312 is angled relative to the mounting surface 311, and the angle between the two surfaces may be a variety of angles including those between 10 and 30 degrees, between 15 and 30 degrees, and between 10 and 25 degrees, and further including 10, 25, and 30 degrees. The angle leads to a peripheral wall for the wedge that tapers from its highest height at its top to its lowest height at its bottom as viewed after implantation.

FIG. 3D illustrates the wedge 300A that includes a mounting surface 311A and a bone facing surface 312A. The illustrated wedge 300A includes the central hole 314 and fastener holes 319 passing through the body 310A. The body 310A, the mounting surface 311A, and the bone facing surface 312A are similar to the body 310, the mounting surface 311, and the bone facing surface 312 with the addition of anchoring holes 314A. The anchoring holes 314A pass through the body 310A from the bone facing surface 312A to the mounting surface 311A. Each of the anchoring holes 314A are configured to align with a respective mounting hole 114B-114E of the baseplate 100 or 100A so that an anchoring mechanism may pass therethrough and attach to the patient's bone. Although 4 anchoring holes 314A are illustrated, the number of holes may range from 2-4 or another number to match the number or lower of the mounting holes 114B-114E present in the baseplate 100 or 100A. The anchoring holes 314A provide additional anchoring beyond just the stem 120 passing through the central hole 314.

In at least one alternative embodiment, the fastening holes 319 are omitted from the wedge 300A. In a further embodiment, the anchoring holes 314A include threads along at least a portion of the length of the opening to being completed threaded for engagement of the anchoring mechanism. FIG. 3E illustrates the wedge 300/300A attached to the baseplate 100B, which could be substituted for by the other baseplates discussed in this disclosure.

In at least one embodiment, the angle between the mounting surface and the bone facing surface of the wedge is 10 degrees, 25 degrees, between 10 and 30 degrees, between 15 and 30 degrees, and/or between 10 and 25 degrees.

In at least one embodiment, the illustrated wedges are integrated into the baseplate as oppose to being a separate component. In an alternative embodiment, the wedge is affixed to the baseplate to form one structure to implant in the patient. In at least one embodiment to the other wedge embodiments, both the wedge and the baseplate are manufactured from metal for implantation on the glenoid bone.

In at least one embodiment, the 10-degree posterior angulation of the wedges facilitate the correction of any bony deformity of the glenoid by reconstruction of the idealized glenoid version in the anterior-posterior and sup-inf planes. The wedges mitigate the need for removal for a revision to occur. In at least one embodiment, the glenoid component in traditional TSR with 7-degree anterior build-up may improve anterior stability.

FIGS. 4A-4F illustrate the partial wedge 400 that generally has a crescent shape to it. The illustrated partial wedge 400 includes a body 410 having three fastener holes 419 although in an alternative embodiment the number of fastener holes 419 may be two. The fastener holes 419 are configured to align with the same number of recessed cavities 119 present in the baseplate 100A. The presence of the fastener holes 419 allows for the partial wedge 400 to be secured to the baseplate 100A prior to insertion in the patient. Although not illustrated and in an alternative embodiment, the fastener holes may include a shoulder on to which the fasteners may abut as oppose (similar to should 315 in FIG. 3C) to the outer surface.

The body 410 includes a central opening 414 through which the stem 120 of the baseplate may pass through for anchoring the system to the patient's bone as illustrated in FIGS. 4D-4G. In at least one embodiment, the central opening 414 has an interference fit with the stem 120.

The partial wedge body 410 includes a mounting surface 411 onto which the attached baseplate abuts and/or is against as illustrated, for example, in FIG. 4E. The opposed surface to the mounting surface 411 is a bone facing surface 412 that is designed to abut and/or be against the patient's bone. The bone facing surface 412 is angled to the mounting surface 411, and the angle between the two surfaces may be a variety of angles similar to the full wedge 300. The angle leads to a peripheral wall for the partial wedge 400 that tapers from its highest height at its top to a tapered edge 413 at its bottom as viewed after implantation. In at least one embodiment, the tapered edge 413 is approximately 0.25 mm thick.

As illustrated in FIGS. 4D and 4F, two of the mounting holes 114A-114D (depending on alignment) are available for insertion of one or more attachment mechanisms 130 (not illustrated) for additional anchoring of the system to the patient's bone. FIG. 4E illustrates how the tapered edge 413 will overlap with the notch 112′, while still allowing access to the notch 112′ to facilitate removal of the baseplate and the wedge with it. FIG. 4F illustrates the presence of fasteners 430 in the partial wedge 400.

FIG. 4G illustrates the partial wedge 400A, which like the partial wedge 400 includes a body 410A, a mounting surface 411A, a bone facing surface 412A, a central opening 414, and fastener holes 419. The body 410A, the mounting surface 411A, and the bone facing surface 412A are similar to the body 410, the mounting surface 411, and the bone facing surface 412 with the addition of anchoring holes 414A. The anchoring holes 414A pass through the body 410A from the bone facing surface 412A to the mounting surface 411A. In at least one embodiment, the anchoring holes 414A are configured to align with a respective number of mounting holes 114B-114E of the baseplate 100 or 100A so that an anchoring mechanism may pass therethrough and attach to the patient's bone. The anchoring holes 414A provide additional anchoring beyond just the stem 120 passing through the central hole 414.

In at least one alternative embodiment, the fastening holes 419 are omitted from the partial wedge 400A. In a further embodiment, the anchoring holes 414A include threads along at least a portion of the length of the opening to being completed threaded for engagement of the anchoring mechanism.

In at least one embodiment, the wedges 300, 300A, 400, and 400A are made, for example, of the same material as the baseplate to be attached to it, a hydroxyapatite-coated (HA-coated) substrate and/or porous material.

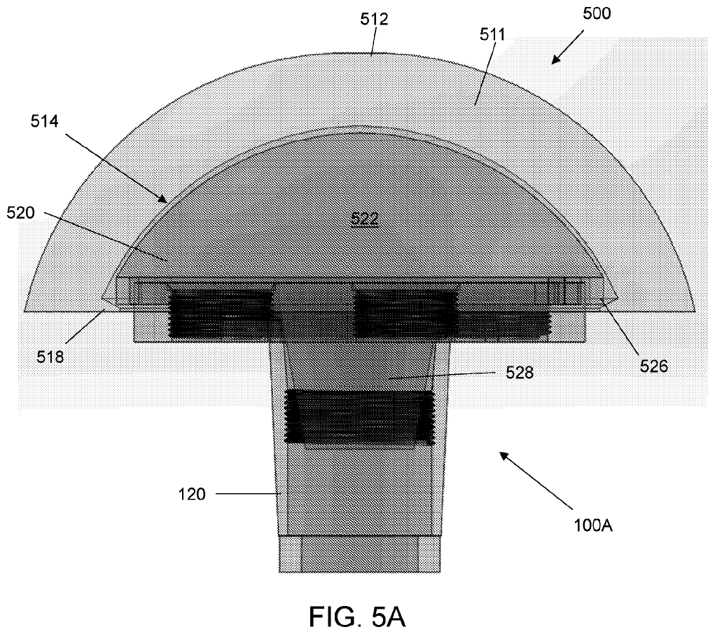

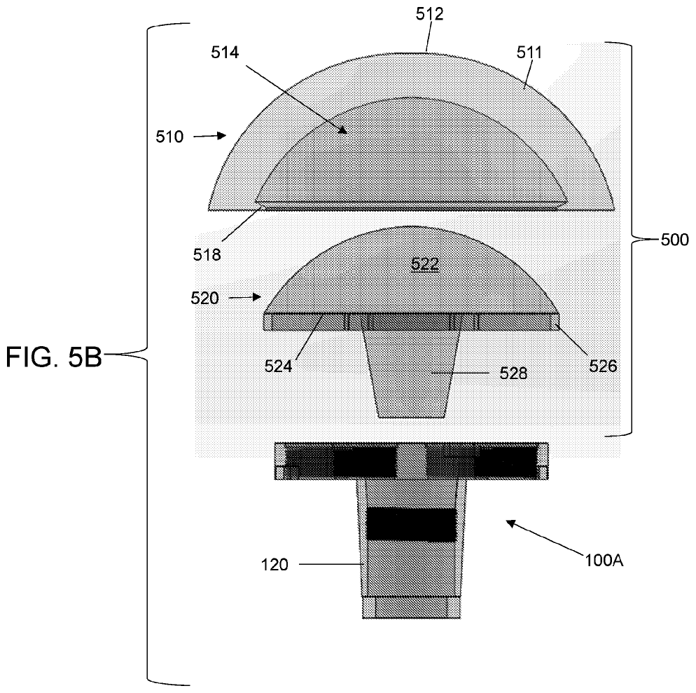

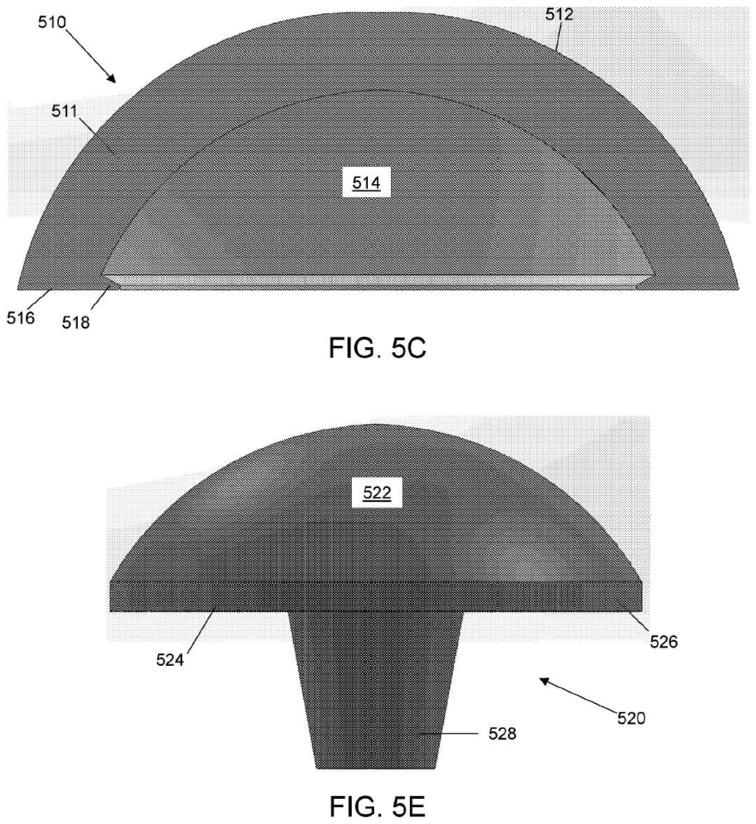

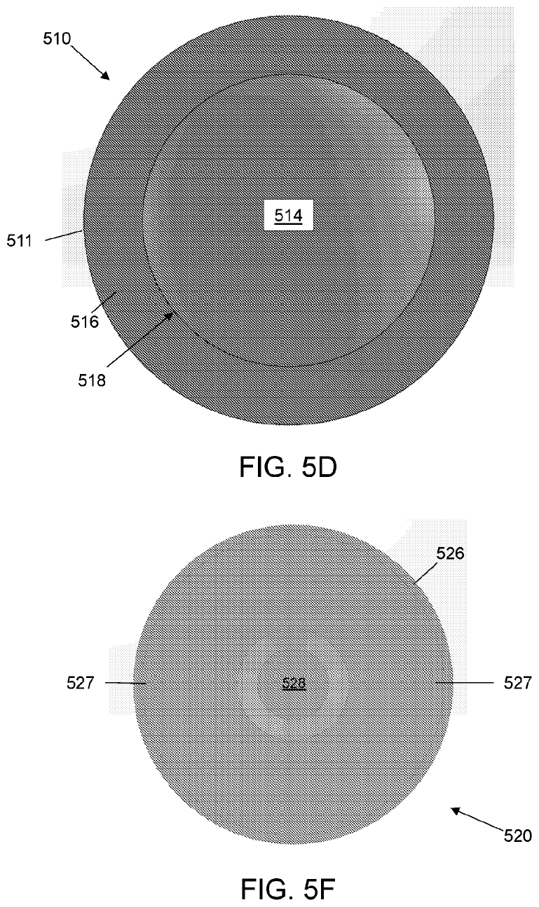

FIGS. 5A-5F and 6A-6K illustrate two examples of a modular humeral component 500, 500A that is a dual mobility humeral head for a ta-TSR. The modular humeral component 500, 500A includes a humeral shell 510, 510A and an inner head 520, 520A that is configured to be inserted and secured in the humeral shell 510, 510A. The shell 510, 510A is able to slide and/or rotate relative to the inner head 520, 520A, and in at least one embodiment illustrated in FIG. 5A the movement is limited by an interior ledge 518 of the shell 510. In at least one embodiment, the shell 510, 510A and the inner head 520, 520A are hemi-spherical. The modular humeral component 500, 500A may be attached to the above-described baseplates or a humeral stem, both of which are examples of a shoulder base. Based on this disclosure, a person having ordinary skill in the art should appreciate reference to attachment to a baseplate in connection with FIGS. 5A-6K could be replaced by a humeral stem.

FIG. 5A illustrates a phantom, cross-sectional view of the modular humeral component 500 installed on the baseplate 100A, while FIG. 5B illustrates an exploded view of the assembly illustrated in FIG. 5A.

As illustrated in FIGS. 5A-5D, the shell 510 includes a body 511 having a generally hemispherical (or convex) surface 512 and defining an interior cavity 514 into which the inner head 520 is inserted. Along the interior side of the bottom 516 of the shell body 511 is an interiorly facing ledge 518 configured to fit under a dome of the inner head 520. In at least one embodiment, the ledge 518 encircles the cavity 514. In at least one embodiment, the ledge 518 includes a tapered structure and/or beveled upper surface, which assists with compression and engagement around the inner head 520. In at least one embodiment, when the modular component is attached to the baseplate, the ledge 518 has a clearance of approximately 1 mm from the peripheral side of the baseplate, which allows for some sliding along an arc of (or pivoting over) the inner head 520. In a further embodiment, the gap between the ledge 518 and the baseplate is smaller to further reduce the arc sliding movement while still allowing for the rotational (or spinning) movement. The exterior surface is configured to interact with a concave surface of a modular glenoid component like that discussed in the previously identified applications and pending PCT Application No. PCT/US2022/026940.

The illustrated inner head 520 includes a dome 522, a base 524, an optional flange 526, and an optional plug 528 for insertion into a receiving cavity 120 of the shoulder base implanted into the patient's humeral bone. In at least one embodiment, the flange 526 is omitted. In at least one embodiment, the exterior bottom peripheral edge of the inner head is beveled to match the beveling of the shell ledge 516. In at least one embodiment, the plug 528 is tapered in a direction from the base to its free end (or tip) as illustrated, for example, in FIG. 5E.

As illustrated in FIGS. 5A-5B and 5E-5F, the flange 526 depends (or extends) from the base 524 such that it can fit over the baseplate. In at least one embodiment, the flange 526 includes protrusions 527, which are configured to engage notches 112, respectively, of the baseplate. Although FIG. 5A illustrates the flange 526 extending part way down the baseplate 100A, in at least one embodiment the flange 526 will extend further down the peripheral side of the baseplate 100A. In a further embodiment, the flange 526 is omitted while keeping the protrusions 527 for engagement of the notches 112. In at least one embodiment, each protrusion 527 has an interference fit with a respective notch 112.

In at least one embodiment, the inner head 520 will have a centrally located plug 528 that is capable of being manually offset or is physically offset from the axial center to allow best coverage of the proximal humeral anatomic neck and metaphysis. The physical offset includes an eccentrically located plug, which when the modular humeral component is rotated provides different amounts of coverage to the humeral side.

In at least one embodiment, the outer shell 510 and/or the inner head 520 will include protrusions extending outwardly from their exterior peripheral sides to allow for an instrument to be used to pry the outer shell 510 and/or the inner head 520 from the humeral base. In an alternative embodiment, the outer shell 510 and/or the inner head 520 will include slots along their bottom structure that are configured to align with notches 112 and/or attachment points 112′ of the baseplate.

FIG. 6A illustrates an exploded, cross-sectional view of the modular humeral component 500A that includes a shell (or outer poly shell) 510A and an inner head (or humeral head base) 520A. FIGS. 6B and 6C illustrate a top perspective view and side view of the shell 510A. FIGS. 6D-6G illustrate a top view, a perspective view, a side view, and a bottom view of the inner head 520A. FIGS. 6H-6K illustrate the humeral head 520A assembled with a bottom perspective view, a side view, a side phantom view, and a bottom view, respectively.

As illustrated in FIGS. 6A-6C, the shell 510A includes a body 511A having a generally hemispherical (or convex) surface 512A and defining an interior cavity 514A into which the inner head 520A is inserted. The exterior surface 512A is configured to interact with a concave surface of a modular glenoid component like that discussed in the previously identified applications and PCT Application No. PCT/US2022/026940. The interior cavity 514A includes a concave section 5142A into which the dome 522A of the inner head 520A fits with the outer peripheral edge including an engagement channel 529 that engages an engagement section 5144A with a ring protrusion 518A of the interior cavity 514A as illustrated in FIGS. 6A and 6J. The engagement surfaces 518, 529 are complementary and in an alternative embodiment are reversed. The engagement channel of the inner head 520A includes a channel 529 with shoulders on either side into which the ring protrusion 518A fits to secure the two pieces together. In at least one embodiment, the bottom section 5146A of the interior cavity 514A is large enough to fit over the baseplate in a manner similar to the previously discussed flange. In a further optional embodiment, the bottom section 5146A includes an inwardly tapered wall 51462A as illustrated in FIGS. 6A, 6H, 6J, and 6K, which tapered wall provides a substantially even thickness for the outer shell in around that section of the cavity. In a further embodiment, the taper wall 51642A may assist with insertion of the inner head 520A and over the baseplate 100C, particularly in the situation where the inner head 520A has been implanted and the outer shell 510A is being replaced. In at least one embodiment, the bottom section 5146A has a diameter for its opening that is just slightly larger than the baseplate diameter. In an alternative embodiment, the bottom section 5146A is omitted from the outer shell 510A leaving the bottom of the outer shell to abut or rest substantially against the baseplate.

FIGS. 6A-6C also illustrate an alternative embodiment for the body 511A that includes a beveled outer edge 5112A along the bottom of the body 511A. The beveled edge 5112A reduces (or eliminates) loading pressure along that edge that could lead to fatigue and/or failure resulting in deterioration of the outer shell and possibly breaking off pieces from the shell edge into the patient's body. In at least one embodiment, the beveled or arcuate edge is added to the outer shell 510 illustrated in FIGS. 5A-5C and 5D.

The illustrated inner head 520A includes a dome 522A, a base 524A, and a plug 528A for insertion into a receiving cavity of the shoulder base embedded into the patient's humeral bone. In at least one embodiment as illustrated in FIGS. 6A, 6E, and 6F, the plug 528A may include a beveled surface where it extends from the base 524A.

FIGS. 6A, 6E, and 6F illustrate the interface to engage the attached shell. The illustrated interface includes a channel 529 that is formed on the peripheral outside of the base 524A. Above and below the channel 529 are shoulders. In at least one embodiment, the channel and shoulders form a groove. The channel 529 although illustrated as having a concave surface when viewed in a cross-section, see, e.g., FIG. 6A, other cross-sections are possible while having the channel 529A and the protrusion 518A of the shell having complementary shapes to each other. The illustrated shape allows for the shell 510A to rotate relative to the inner head 520A. In an embodiment where there is a small gap between the channel 529A and the protrusion 518A, then the shell 510A may move in the radial direction relative to the inner head 520A.

FIGS. 6A and 6E-6K illustrate the presence of a Morse taper for the plug 528A that includes a beveled tip 5282A and also a shaped interface 5284A between the post 528A and the base 524A. In at least one embodiment, the shaped interface 5284A is beveled, while in an alternative embodiment it arcuate shaped. The baseplate 100C illustrated in FIG. 6A includes a complementary cavity 124C that includes a tapered surface to match the Morse taper.

In at least one embodiment for either of the described modular humeral components, the inner head provides a universal connection between the baseplate and different size of shells. For example, this allows for a reduced inventory of humeral components for TSR while providing flexibility.

In at least one embodiment, the largest humeral head diameter at a minimum thickness is 58 mm×18 mm. Examples of humeral head diameters include 38 mm to 52 mm, and more particularly 38 mm, 40 mm, 42 mm, 44 mm, 46 mm, 48 mm, 50 mm, and 52 mm with thicknesses being 15 mm-18 mm or 18 mm-21 mm.

In an alternative embodiment, the outer shell 510, 510A is a dual mobility shell. Dual mobility reduces the presence of polyethylene and other wear particles in the joint and between the shell 510, 510A and the inner head 520, 520A compared to traditional approaches, which helps mitigate loosening and device failure. Dual mobility also facilitates improved range of motion for the patient. Both of these lead to better patient outcomes. In an alternative embodiment, the outer shell 510 is removable from the inner head 520 to facilitate replacement, for example when the outer shell 510 is worn down from biomechanical movement about the shoulder joint.

In at least one embodiment, the inner head 520 will be made from Co-Cr while the outer shell 510 will be made from high-density polyethylene, which will avoid the issue of having metal components rub against other metal components, which could lead to a faster wear on the components and potentially create loose metal shavings within the shoulder socket. Additional examples of material for the shell include UHMWPE-highly crosslinked, a Vitamin E doped polyethylene, and Vitamin E HDPE. Additional examples of material for the inner head include Ti6Al4V, a titanium-aluminum alloy, and cobalt-chromium. In a further embodiment, the inner head 520A may be manufactured and bonded to the outer shell 510A to provide a single fabricated component. In a further embodiment, the bonding is present in the channel 529 and/or on the protrusion 518A.

Although particular materials have been identified for particular components and structural elements, one of ordinary skill in the art will appreciate that other materials may be substituted without departing from the scope of the invention. In at least one embodiment, modules attached to the humeral side and the glenoid side will not both be the same material at the point of interaction.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

As used above “substantially,” “generally,” “approximately,” and other words of degree are relative modifiers intended to indicate permissible variation from the characteristic so modified particularly when relating to manufacturing and production tolerances. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

1. A modular humeral component for use in a modular shoulder prosthesis system having a shoulder base with or without notches and/or attachment points, the modular humeral component comprising:
an inner head having
a base,
a dome on said base, and
an optional plug extending away from said base; and
a shell configured to fit over said inner head, said shell having
a body having an exterior surface configured for engagement with a concave surface of a glenoid component, said body defining an interior cavity configured to receive said dome, and
an interiorly facing engagement means for attachment of said shell to said inner head where said engagement means is in the interior cavity.

2. The modular humeral component according to claim 1, wherein said shell fits over the shoulder base and an inner diameter of an opening of the interior cavity is slightly larger than a diameter of the shoulder base and/or
wherein said shell includes an outer lower edge that is beveled, convex, or arcuate-shaped.

3. (canceled)

4. The modular humeral component according to claim 1, wherein said dome is a hemi-spherical dome and a surface of said interior cavity of said shell is hemi-spherical,
said shell is configured to slide relative about said dome to allow for dual mobility between said shell and said inner head such that any pivoting between them is limited by a ledge or said shell is configured to freely rotate relative to said inner head.

5. The modular humeral component according to any claim 1, wherein said inner head is bonded to said shell.

6. (canceled)

7. The modular humeral component according to claim 1, wherein said inner head includes cobalt-chromium and said shell includes a high-density polyethylene.

8. The modular humeral component according to claim 1, wherein
said inner head includes Ti6Al4V, a titanium-aluminum alloy, or cobalt-chromium, and/or
said shell includes UHMWPE-highly crosslinked, a Vitamin E doped polyethylene, or Vitamin E HDPE.

9. The modular humeral component according to claim 1, wherein said engagement means includes a ring protrusion that engages a channel around a peripheral lower edge of said base.

10. (canceled)

11. The modular humeral component according to claim 9, wherein said base further includes a shoulder above said channel and a lower shoulder below said channel that together said shoulders define a groove as said channel.

12. The modular humeral component according to claim 9, wherein said ring protrusion and said channel have complementary shapes configured to allow for rotation of said shell relative to said inner head.

13. The modular humeral component according to claim 9, wherein said ring protrusion and said channel have an approximate semi-circular cross-section or a partial elliptical shape.

14. (canceled)

15. (canceled)

16. The modular humeral component according to claim 1, wherein
an opening of said interior cavity includes a beveled or tapered edge with a reducing diameter from its outer edge and optionally a diameter at a top of said beveled or tapered edge is slightly larger than a diameter of the shoulder base onto which said modular humeral component is configured to fit over or
said inner cavity includes a semi-spherical section configured to receive said dome of said inner head where said semi-spherical section is interior to said engagement means and an optional beveled section prior to said engagement means.

17. (canceled)

18. The modular humeral component according to claim 1, wherein said engagement means includes a ledge configured to hook over said inner head or a ledge with a top surface beveled in a direction of its free edge.

19. The modular humeral component according to claim 18, wherein a bottom exterior edge of said inner head is beveled to correspond to the bevel of said ledge.

20. (canceled)

21. The modular humeral component according to claim 1, wherein after said modular humeral component is installed on the shoulder base, a gap between a free edge of said ledge and the shoulder base is approximately 1 mm.

22. A modular component for use in a modular shoulder prosthesis system having a shoulder base, such as a baseplate or a humeral stem, with notches and/or attachment points, the modular humeral component comprising:
an inner head having
a base, and
a dome on said base; and
a shell configured to fit over said inner head, said outer shell having
a body having a convex surface, said body defining an interior cavity configured to receive said dome, and
an interiorly facing ledge along a bottom of said body and/or a ring protrusion that is parallel to a bottom of said body configured to engage a complimentary channel below said dome of said inner head.

23. (canceled)

24. The modular component according to claim 22, wherein a top surface of said ledge is beveled in a direction of its free edge.

25. The modular component according to claim 24, wherein a bottom exterior edge of said inner head base is beveled to correspond to the bevel of said ledge.

26. The modular component according to claim 22, wherein said inner head includes a plug is-tapered in a direction from said base to its free tip to define a Morse taper.

27. (canceled)

28. The modular component according to claim 22, wherein said dome is a hemi-spherical dome and said interior cavity of said shell is hemi-spherical,
said shell is configured to slide relative about said dome to allow for dual mobility between said shell and said inner head such that any pivoting between them is limited by said ledge or said shell is configured to freely rotate relative to said inner head.

29. (canceled)

30. (canceled)

31. The modular component according to claim 22, wherein said inner head includes cobalt-chromium and said shell includes a high-density polyethylene.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A modular shoulder prosthesis system comprising:
a humeral stem having a mounting surface into which a receiving cavity extends; and
a central stem extending from said mounting surface;
said modular component according to claim 1 configured to be removably attached to said mounting surface, and
wherein said humeral stem is capable of attachment to different modular components to facilitate both traditional anatomic total shoulder replacement and reverse total shoulder replacement with a change in the modular component.

54. (canceled)
